(19) United States
(12) Patent Application Publication
(10) Pub. No.: US 2023/0027317 A1
(43) Pub. Date: Jan. 26, 2023

(54) NON-HUMAN PRIMATE ALZHEIMER'S DISEASE MODEL ANIMAL AND METHOD FOR PRODUCING SAME

(71) Applicants: RIKEN, Saitama (JP); CENTRAL INSTITUTE FOR EXPERIMENTAL ANIMALS, Kanagawa (JP)

(72) Inventors: Erika SASAKI, Kanagawa (JP); Kenya SATO, Kanagawa (JP); Wakako KUMITA, Kanagawa (JP); Hiroki SASAGURI, Saitama (JP); Takaomi SAIDO, Saitama (JP); Kenichi NAGATA, Saitama (JP); Takashi YAMAMOTO, Hiroshima (JP); Tetsushi SAKUMA, Hiroshima (JP)

(21) Appl. No.: 17/784,942

(22) PCT Filed: Dec. 18, 2020
(86) PCT No.: PCT/JP2020/047548

§ 371 Date: Jun. 13, 2022

Related U.S. Application Data
(60) Provisional application No. 62/951,498, filed on Dec. 20, 2019.

(51) Int. Cl. A01K 67/027

(52) U.S. Cl. CPC A01K 67/0276; A01K 2217/075; A01K 2227/106; A01K 2267/0312

(57) ABSTRACT

Provided is an animal model that can replicate a disease state of human AD. A non-human primate model animal of Alzheimer's disease includes the PSEN1 gene in which a site related to splicing of exon 9 is made deficient.

### TECHNICAL FIELD

The present invention relates to a non-human primate model animal of Alzheimer's disease and a method for producing the model animal.

### BACKGROUND ART

Alzheimer's disease (AD) is a major cause of dementia, with the number of patients with AD anticipated to exceed 50 million worldwide. However, no effective measures have been established to prevent or treat AD, which is due in part to a lack of useful animal models. Some mice models have been established, but cannot reflect human AD disease states in most parts due to biological differences from primates.

### CITATION LIST

### Non-Patent Literatures

Non-Patent Literature 1
Sasaguri H. et al. APP mouse models for Alzheimer's disease preclinical studies. EMBO J 1; 36(17):2473-2487, doi: 10.15252/embj.201797397 (2017).

Non-Patent Literature 2
Saito T. et al. Single App knock-in mouse models of Alzheimer's disease. Nat Neurosci. 17(5):661-663. doi:10.1038/nn.3697 (2014).

Non-Patent Literature 3
Oakley H et al. Intraneuronal beta-amyloid aggregates, neurodegeneration, and neuron loss in transgenic mice with five familial Alzheimer's disease mutations: potential factors in amyloid plaque formation. J Neurosci. 26(40):10129-10140. doi:10.1523/JNEUROSCI.1202-06. (2006).

### SUMMARY OF INVENTION

### Technical Problem

An objective of the present invention is to provide a world's first non-human primate AD model.

### Solution to Problem

To attain the above objective, the inventors performed various experiments and found that mutant PSEN1 marmoset individuals whose 3′-splice site (acceptor site) of exon 9 in the PSEN1 gene (that encodes presenilin 1 protein) was partly or completely deleted by Transcription Activator-Like Effector Nuclease (TALEN) in marmoset embryos and individuals could replicate a pathological status observed in human AD patients. On the basis of this finding, the inventors completed the present invention. Specifically, the present invention includes the following features.

(1) A non-human primate model animal of Alzheimer's disease, including: the PSEN1 gene in which a site including at least a 5′- or 3′-splice site of exon 9 and being related to splicing of exon 9 is deficient.

(2) The non-human primate model animal of Alzheimer's disease described in (1), wherein the non-human primate is a marmoset.

(3) A method for producing a non-human primate model animal of Alzheimer's disease, the method including: making a site of the PSEN1 gene deficient by a genome-editing technology, the site including at least a 5′- or 3′-splice site of exon 9 in the PSEN1 gene and being related to splicing of exon 9.

(4) The method described in (3), wherein the site including at least the 5′- or 3′-splice site of exon 9 in the PSEN1 gene and being related to splicing of exon 9 is made deficient in an ovum.

(5) The method described in (3) or (4), wherein the non-human primate is a marmoset.

In another aspect, the present invention includes the following features:

(6) A non-human primate model animal of Alzheimer's disease including: the PSEN1 gene in which an acceptor site of exon 9 is deficient.

(7) The non-human primate model animal of Alzheimer's disease described in (6), wherein the non-human primate is a marmoset.

(8) A method for producing a non-human primate model animal of Alzheimer's disease, the method including: making an acceptor site of exon 9 in the PSEN1 gene deficient by a genome-editing technology.

(9) The method described in (8), wherein the acceptor site of exon 9 in the PSEN1 gene is made deficient in an ovum.

### Advantageous Effects of Invention

In accordance with an aspect of the present invention, it is possible to replicate a human AD disease state.

### BRIEF DESCRIPTION OF DRAWINGS

FIG. 1 is a conceptual view schematically illustrating an example of Platinum TALEN, which is utilizable in the present invention, targeting the 3′-splice site of exon 9 in a marmoset. In FIG. 1, the regions surrounded by the quadrangular frames correspond to TALEN target sites and exons in the marmoset PSEN1 gene, and the numbers surrounded by the quadrangular frames indicate exon numbers. The 3′-splice site (“AG”) of exon 9 is located upstream of exon 9.

a of FIG. 2 shows a result of surveyor assay carried out, in an Example, after injection of TALEN in the marmoset fertilized ova (three left columns; “PC” on the right side is a positive control, and “NC” on the right side is a negative control). b of FIG. 2 is a view schematically illustrating an outline of TALEN injection and subsequent PSEN1 mRNA analysis, which were carried out in the Example. c of FIG. 2 shows a result of reverse transcription PCR (RT-PCR) on the fertilized ova after TALEN injection, which was carried out in the Example.

a of FIG. 3 is a photograph of a neonate of a marmoset AD model in accordance with an aspect of the present invention, which was obtained in an Example. b of FIG. 3 shows a result of surveyor assay carried out using genomic DNAs extracted from cord blood of the neonate. c of FIG. 3 shows a result of PCR carried out using RNAs extracted from hair roots of the marmoset neonate and a wild-type marmoset.

FIG. 4 shows a result of quantification of two kinds of amyloid β proteins in the fibroblasts collected from ear tissues of mutant marmosets (8 month old) obtained in an Example.

a of FIG. 5 is a photograph of a neonate of a marmoset AD model in accordance with an aspect of the present invention, obtained in Example 4. b of FIG. 5 shows a result of surveyor assay carried out using genomic DNAs extracted from cord blood of the neonate. c of FIG. 5 shows sequences of wild-type and mutant neonate genomes around the 3′-splice site of exon 9.

a of FIG. 6 shows a result of Western blot analysis carried out using homogenates of fibroblasts and antibodies to PS1 proteins (left: N-terminal fragment (PS1 NTF), right: C-terminal fragment (PS1 CTF)). b of FIG. 6 shows a result of quantification of two kinds of amyloid β proteins in the fibroblasts collected from the ear tissues of the mutant marmosets (8 month old) obtained in the Example. All the fibroblasts were collected from the ear tissues of the mutant marmosets (8 month old) obtained in Example 4.

### DESCRIPTION OF EMBODIMENTS

The present invention relates to non-human primate models of AD. There is no particular limitation on the kind of non-human primate, provided that the primate can have Alzheimer's disease. Examples of the primate encompass Strepsirrhini (e.g., lemurs, lorises, and galagos) and Haplorhini (e.g., tarsiers, spider monkeys, capuchin monkeys, marmosets, guenons, colobi, and anthropoid apes). However, this is not limitative. In a preferred aspect, examples of the non-human primates encompass guenons (e.g., rhesus monkeys and crab-eating macaques) and marmosets (e.g., common marmosets and black-eared marmosets). A marmoset AD model in accordance with an aspect of the present invention is advantageous for AD research for the following reasons: marmosets have a genetic background and a brain structure close to those of humans; marmosets exhibit human-like cognitive behaviors associated with prefrontal cortex; marmosets are able to communicate by visual and auditory information: marmosets have a small body size (e.g., 350 to 500 g); marmosets have a relatively short gestation period (145 days) and their reproductive efficiency is high (40 to 80 neonates per female); and marmosets have a relatively long life span (10 to 13 years in captivity). Marmosets are advantageous also for the following reasons: marmosets exhibit deposition of amyloid β protein with aging starting around 7 years; and the marmoset amyloid β protein has a sequence identical to that of human amyloid β protein.

A marmoset AD model in accordance with an aspect of the present invention has the PSEN1 gene in which a splice site of exon 9 is destroyed. The destruction of the splice site may be carried out by a known genome-editing technology, examples of which encompass CRISPR-Cas9 system and Transcription Activator-Like Effector Nuclease (TALEN). In an aspect, the destruction is preferably carried out by Platinum TALEN. It has been reported that a deletion mutation in exon 9 or a point mutation at a splice site of exon 9 in the PSEN1 gene instigates exclusion of exon 9 in an mRNA due to alternative splicing. The inventors are the first to actually succeed in production of a non-human primate AD model by destruction of this site.

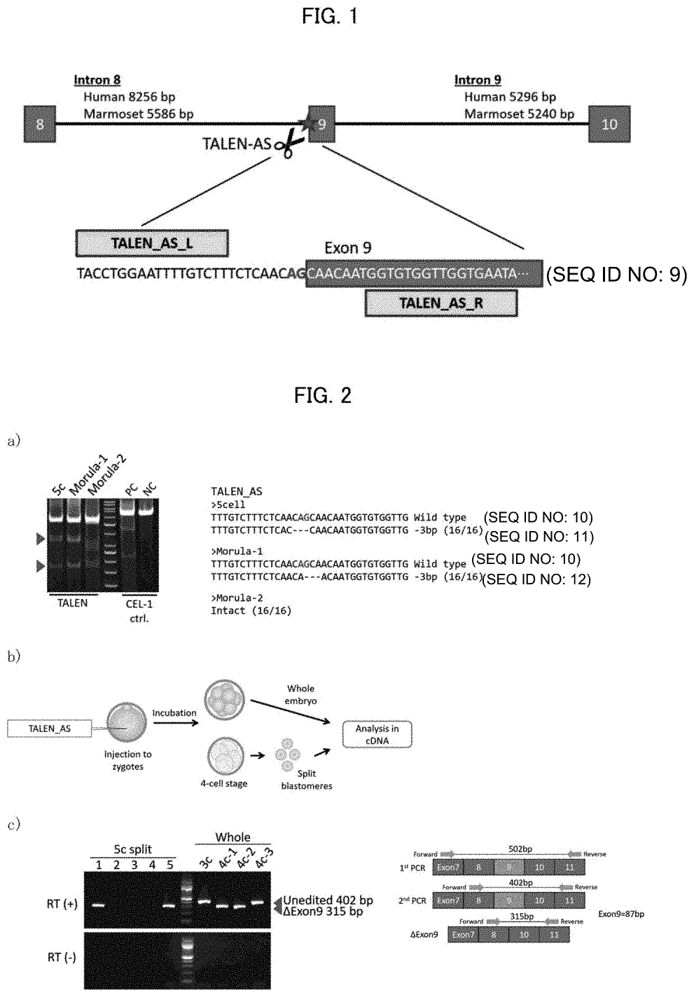

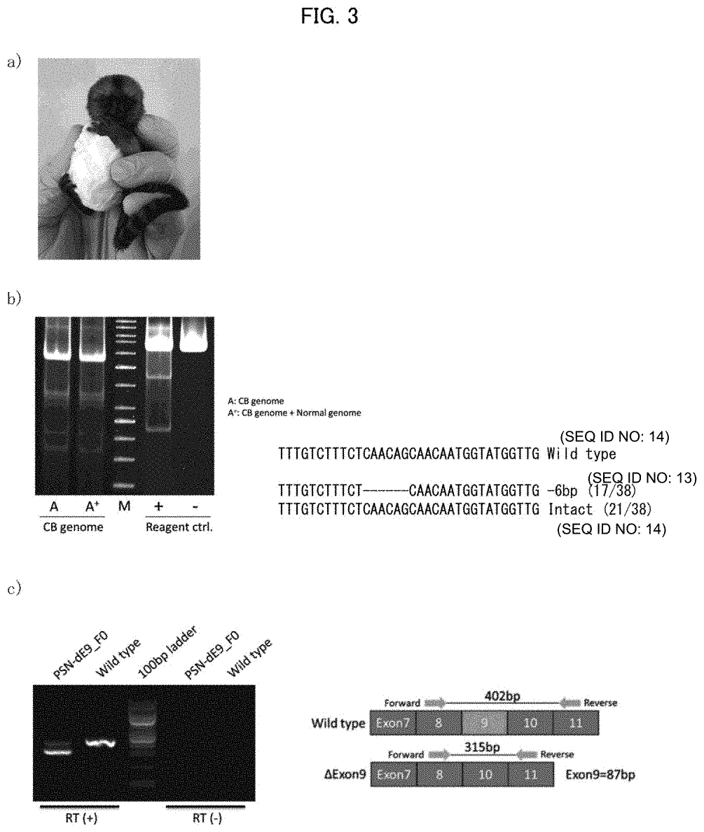

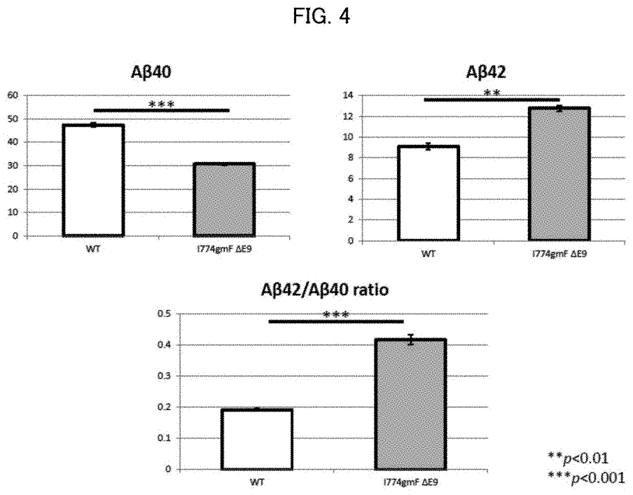

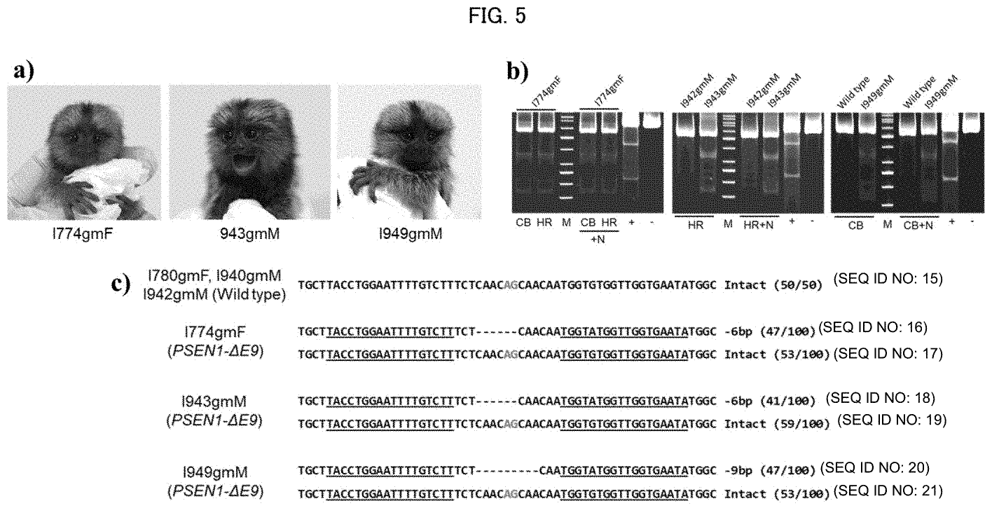

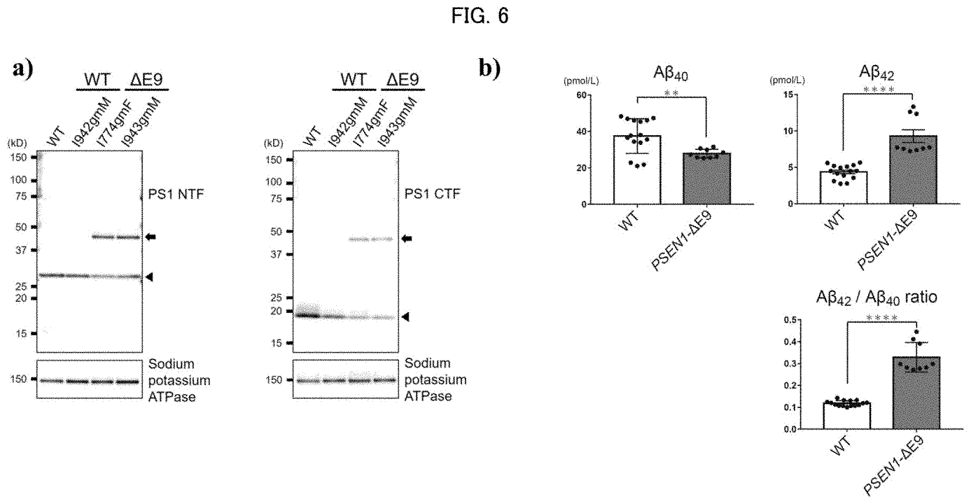

FIG. 1 is a conceptual view of Platinum TALEN targeting the 3′-splice site of exon 9 in the marmoset PSEN1 gene. Each of TALEN_AS_L indicated on the left and TALEN_AS_R indicated on the right in FIG. 1 can be constructed by a known method including use of Platinum Gate TALEN kit. Each of TALEN_AS_L and TALEN_AS_R is a TALEN target site. A region of a sequence surrounded by a quadrangular frame corresponds to exon of the marmoset PSEN1 gene. Bold-faced “AG” adjacent to exon 9 on the upstream side corresponds to the 3′-splice site of exon 9. The 5′-splice site is “GT” (not illustrated) adjacent to exon 9 on the downstream side.

In a preferred embodiment, the expression “3′-splice site of exon 9 in the PSEN1 gene” indicates two nucleotides existing at the 3′-terminal of intron 8 for splicing of exon 8 and exon 9, as shown in FIG. 1. The expression “deficiency of the 3′-splice site of exon 9 in the PSEN1 gene” means functional deficiency of the 3′-splice site. The deficiency is caused by deletion of one or both of the two nucleotides (only nucleotide A, only nucleotide G, or both nucleotides AG) (see FIGS. 2 and 3). In the embodiment, the “deficiency of a site including at least the 3′-splice site of exon 9 in the PSEN1 gene (i.e., the site being related to splicing of exon 9)” is essentially caused by the deletion.

The deficiency is any of (i) partial deletion of the 3′-terminal (including the above-described nucleotides AG) of intron 8 and the 5′-terminal of exon 9, (ii) partial deletion of the 3′-terminal (including the above-described nucleotides AG or nucleotide A) of intron 8, and (iii) partial deletion of the nucleotide G or nucleotides AG and the 5′-terminal of exon 9. The full length of the deletion causing the deficiency may be arbitrarily selected, provided that the deletion includes one or both of the two nucleotides and does not give any effect on splicing of any other exon than exon 9.

In another embodiment different from the preferred embodiment shown in FIG. 1, the deficiency may be caused by deletion of the 5′-splice site of exon 9 in the PSEN1 gene. The meaning of the deficiency in this embodiment can be understood by the preceding two paragraphs in which the following parts are replaced as below.

1. The “3′-splice site” may be replaced with “5′-splice site”.
2. The “exon 8 and exon 9” may be replaced with “exon 9 and exon 10”.
3. The “3′-terminal of intron 8” may be replaced with “5′-terminal of intron 9”.
4. The “5′-terminal of exon 9” may be replaced with “3′-terminal of exon 9”.
5. The “nucleotide A” may be replaced with “nucleotide G”, the “nucleotide G” may be replaced with “nucleotide T”, and “nucleotides AG” may be replaced with “nucleotides GT”.

In another embodiment different from the above two embodiments, the “deficiency of a site including at least the 5′-splice site or the 3′-splice site of exon 9 in the PSEN1 gene (the site being related to splicing of exon 9)” can be caused by deletion of a branch site existing in intron. The intron can be either of or both intron 8 and intron 9 in the PSEN1 gene. It is known that the branch site exits near the 3′-terminal in the intron and is followed by, on its downstream side, a polypyrimidine region (repetition of Py). In an aspect of the present invention, the branch site can be a site including nucleotide A located 20 to 40 (said to be 21 to 34 nucleotides) nucleotides upstream of the 3′-splice site. The deletion of the branch site means deletion of at least the above-described nucleotide A.

Note that a genome of a marmoset AD model in accordance with an aspect of the present invention may have all of or any two of the above-described three different deletions.

A method for producing a marmoset AD model in accordance with an aspect of the present invention is, for example, as follows. TALEN mRNAs targeting the 3′-splice site of exon 9 in the marmoset PSEN1 gene are introduced into the nuclei of marmoset fertilized ova. The resulting products may be subcloned, followed by surveyor assay and sequencing analysis. Alternatively, only embryos in which a target site including the 3′-splice site is deficient may be collected. The deficiency can be induced at a high possibility, e.g., two out of three. After the introduction of TALEN, RT-PCR and cDNA sequencing can be carried out to confirm exclusion of exon 9 in the PSEN1 mRNA. According to this method, there is no wild-type sequence in an RNA extracted from a 4-cell-stage fertilized ovum or a single blastomere, and the 3′-splice site can be made deficient in a biallelic manner.

Preferably, TALEN mRNAs are introduced into ova, and then the ova are fertilized. After fertilization, the fertilized ova are developed to a 6-cell stage or more. Then, the fertilized ova can be transferred to surrogate mothers. The introduction of TALEN does not cause any distinct adverse reaction in pregnancy or following development. In this manner, it is possible to obtain marmosets carrying, e.g., a 6-bp deficiency including the 3′-splice site in a heterozygous state. It is possible to confirm exclusion of exon 9 by carrying out, on mRNAs from the obtained marmoset AD models (e.g., the mRNAs extracted from hair roots of the marmosets), RT-PCR and then cDNA sequencing.

As indicated in the later-described Examples, a marmoset AD model in accordance with an aspect of the present invention exhibits an increase of Aβ₄₂/Aβ₄₀ ratio compared to that of a wild-type marmoset, where Aβ₄₂ is amyloid β protein Aβ₄₂ and Aβ₄₀ is amyloid β protein Aβ₄₀. For example, a marmoset AD model in accordance with an aspect of the present invention has a value of Aβ₄₂/Aβ₄₀ that is 1.4-fold or more, 1.6-fold or more, 1.8-fold or more, or 2-fold or more greater than that of the wild-type.

### EXAMPLES

Examples are indicated below. However, the present invention is not limited to these Examples.

All the animal experiments described below were conducted in accordance with the guidelines for animal experimentation as determined by the Central Institute for Experimental Animals on the basis of “the Act on Welfare and Management of Animals”, “Standard relating to the Care and Keeping and Reducing Pain of Laboratory Animals” and “Fundamental Guidelines for Proper Conduct of Animal Experiment and Related Activities in Academic Research Institutions (notified by the Ministry of Education, Culture, Sports, Science and technology of Japan)”.

Materials and Method

Preparation of TALEN Plasmids

Platinum Gate TALEN Kit (Addgene) was used to prepare TALEN plasmids in accordance with a method described in SCIENTIFIC REPORTS 2013, November 29; 3: 3379. Doi:10.1038/srep03379. This method will be briefly explained below. Platinum Gate TALEN kit (Addgene; cat #1000000043) was used to carry out two-step Golden Gate assembly method, so as to construct Platinum TALEN containing a homodimer-type FokI nuclease domain. The assembled repeat arrays were subsequently inserted into a final destination vector, ptCMV-153/47-VR. TALEN mRNA was synthesized with use of “mMESSAGE mMACHINE T7 Ultra Transcription Kit” (Thermo Fisher Scientific, AM1345). Transcribed mRNA was purified with use of “MEGAclear Transcription Clean-Up Kit” (Thermo Fisher Scientific, AM1908). The full-length sequences of the plasmids thus prepared are indicated as SEQ ID NO: 1 (Left_TALEN) and SEQ ID NO: 2 (Right_TALEN) in the sequence listing.

Sequences of TALEN Vectors

SEQ ID NO: 1
AGAAGGGGCTCCCGCACGCGCCTGCATTGATTAAGCGGACCAACAGAAGG
ATCCCCGAGAGGACATCACATCGAGTGGCAGGTTCCCAACTCGTGAAGAG
TGAACTTGAGGAGAAAAAGTCGGAGCTGCGGCACAAATTGAAATACGTAC
CGCATGAATACATCGAACTTATCGAAATTGCTAGGAACTCGACTCAAGAC
AGAATCCTTGAGATGAAGGTAATGGAGTTCTTTATGAAGGTTTATGGATA
CCGAGGGAAGCATCTCGGTGGATCACGAAAACCCGACGGAGCAATCTATA
CGGTGGGGAGCCCGATTGATTACGGAGTGATCGTCGACACGAAAGCCTAC
AGCGGTGGGTACAATCTTCCCATCGGGCAGGCAGATGAGATGCAACGTTA
TGTCGAAGAAAATCAGACCAGGAACAAACACATCAATCCAAATGAGTGGT
GGAAAGTGTATCCTTCATCAGTGACCGAGTTTAAGTTTTTGTTTGTCTCT
GGGCATTTCAAAGGCAACTATAAGGCCCAGCTCACACGGTTGAATCACAT
TACGAACTGCAATGGTGCGGTTTTGTCCGTAGAGGAACTGCTCATTGGTG
GAGAAATGATCAAAGCGGGAACTCTGACACTGGAAGAAGTCAGACGCAAG
TTTAACAATGGCGAGATCAATTTCCGCTCATAAAAAATCAGCCTCGACTG
TGCCTTCTAGTTGCCAGCCATCTGTTGTTTGCCCCTCCCCCGTGCCTTCC
TTGACCCTGGAAGGTGCCACTCCCACTGTCCTTTCCTAATAAAATGAGGA
AATTGCATCACAACACTCAACCCTATCTCGGTCTATTCTTTTGATTTATA
AGGGATTTTGCCGATTTCGGCCTATTGGTTAAAAAATGAGCTGATTTAAC
AAAAATTTAACGCGAATTAATTCTGTGGAATGTGTGTCAGTTAGGGTGTG
GAAAGTCCCCAGGCTCCCCAGCAGGCAGAAGTATGCAAAGCATGCATCTC
AATTAGTCAGCAACCAGGTGTGGAAAGTCCCCAGGCTCCCCAGCAGGCAG
AAGTATGCAAAGCATGCATCTCAATTAGTCAGCAACCATAGTCCCGCCCC
TAACTCCGCCCATCCCGCCCCTAACTCCGCCCAGTTCCGCCCATTCTCCG
CCCCATGGCTGACTAATTTTTTTTATTTATGCAGAGGCCGAGGCCGCCTC
TGCCTCTGAGCTATTCCAGAAGTAGTGAGGAGGCTTTTTTGGAGGCCTAG
GCTTTTGCAAAAAGCTCCCGGGAGCTTGTATATCCATTTTCGGATCTGAT
CAGCACGTGATGAAAAAGCCTGAACTCACCGCGACGTCTGTCGAGAAGTT
TCTGATCGAAAAGTTCGACAGCGTTTCCGACCTGATGCAGCTCTCGGAGG
GCGAAGAATCTCGTGCTTTCAGCTTCGATGTAGGAGGGCGTGGATATGTC
CTGCGGGTAAATAGCTGCGCCGATGGTTTCTACAAAGATCGTTATGTTTA
TCGGCACTTTGCATCGGCCGCGCTCCCGATTCCGGAAGTGCTTGACATTG
GGGAATTCAGCGAGAGCCTGACCTATTGCATCTCCCGCCGTGCACAGGGT
GTCACGTTGCAAGACCTGCCTGAAACCGAACTGCCCGCTGTTCTGCAGCC
GGTCGCGGAGGCCATGGATGCGATCGCTGCGGCCGATCTTAGCCAGACGA
GCGGGTTCGGCCCATTCGGACCGCAAGGAATCGGTCAATACACTACATGG
CGTGATTTCATATGCGCGATTGCTGATCCCCATGTGTATCACTGGCAAAC
TGTGATGGACGACACCGTCAGTGCGTCCGTCGCGCAGGCTCTCGATGAGC
TGATGCTTTGGGCCGAGGACTGCCCCGAAGTCCGGCACCTCGTGCACGCG
GATTTCGGCTCCAACAATGTCCTGACGGACAATGGCCGCATAACAGCGGT
CATTGACTGGAGCGAGGCGATGTTCGGGGATTCCCAATACGAGGTCGCCA
ACATCTTCTTCTGGAGGCCGTGGTTGGCTTGTATGGAGCAGCAGACGCGC
TACTTCGAGCGGAGGCATCCGGAGCTTGCAGGATCGCCGCGGCTCCGGGC
GTATATGCTCCGCATTGGTCTTGACCAACTCTATCAGAGCTTGGTTGACG
GCAATTTCGATGATGCAGCTTGGGCGCAGGGTCGATGCGACGCAATCGTC
CGATCCGGAGCCGGGACTGTCGGGCGTACACAAATCGCCCGCAGAAGCGC
GGCCGTCTGGACCGATGGCTGTGTAGAAGTACTCGCCGATAGTGGAAACC
GACGCCCCAGCACTCGTCCGAGGGCAAAGGAATAGCACGTGCTACGAGAT
TTCGATTCCACCGCCGCCTTCTATGAAAGGTTGGGCTTCGGAATCGTTTT
CCGGGACGCCGGCTGGATGATCCTCCAGCGCGGGGATCTCATGCTGGAGT
TCTTCGCCCACCCCAACTTGTTTATTGCAGCTTATAATGGTTACAAATAA
AGCAATAGCATCACAAATTTCACAAATAAAGCATTTTTTTCACTGCATTC
TAGTTGTGGTTTGTCCAAACTCATCAATGTATCTTATCATGTCTGTATAC
CGTCGACCTCTAGCTAGAGCTTGGCGTAATCATGGTCATTACCAATGCTT
AATCAGTGAGGCACCTATCTCAGCGATCTGTCTATTTCGTTCATCCATAG
TTGCCTGACTCCCCGTCGTGTAGATAACTACGATACGGGAGGGCTTACCA
TCTGGCCCCAGCGCTGCGATGATACCGCGAGAACCACGCTCACCGGCTCC
GGATTTATCAGCAATAAACCAGCCAGCCGGAAGGGCCGAGCGCAGAAGTG
GTCCTGCAACTTTATCCGCCTCCATCCAGTCTATTAATTGTTGCCGGGAA
GCTAGAGTAAGTAGTTCGCCAGTTAATAGTTTGCGCAACGTTGTTGCCAT
CGCTACAGGCATCGTGGTGTCACGCTCGTCGTTTGGTATGGCTTCATTCA
GCTCCGGTTCCCAACGATCAAGGCGAGTTACATGATCCCCCATGTTGTGC
AAAAAAGCGGTTAGCTCCTTCGGTCCTCCGATCGTTGTCAGAAGTAAGTT
GGCCGCAGTGTTATCACTCATGGTTATGGCAGCACTGCATAATTCTCTTA
CTGTCATGCCATCCGTAAGATGCTTTTCTGTGACTGGTGAGTACTCAACC
AAGTCATTCTGAGAATAGTGTATGCGGCGACCGAGTTGCTCTTGCCCGGC
GTCAATACGGGATAATACCGCGCCACATAGCAGAACTTTAAAAGTGCTCA
TCATTGGAAAACGTTCTTCGGGGCGAAAACTCTCAAGGATCTTACCGCTG
TTGAGATCCAGTTCGATGTAACCCACTCGTGCACCCAACTGATCTTCAGC
ATCTTTTACTTTCACCAGCGTTTCTGGGTGAGCAAAAACAGGAAGGCAAA
ATGCCGCAAAAAAGGGAATAAGGGCGACACGGAAATGTTGAATACTCATA
TTCTTCCTTTTTCAATATTATTGAAGCATTTATCAGGGTTATTGTCTCAT
GAGCGGATACATATTTGAATGTATTTAGAAAAATAAACAAATAGGGGTCA
GTGTTACAACCAATTAACCAATTCTGAACATTATCGCGAGCCCATTTATA
CCTGAATATGGCTCATAACACCCCTTGCTCATGACCAAAATCCCTTAACG
TGAGTTACGCGCGCGTCGTTCCACTGAGCGTCAGACCCCGTAGAAAAGAT
CAAAGGATCTTCTTGAGATCCTTTTTTTCTGCGCGTAATCTGCTGCTTGC
AAACAAAAAAACCACCGCTACCAGCGGTGGTTTGTTTGCCGGATCAAGAG
CTACCAACTCTTTTTCCGAAGGTAACTGGCTTCAGCAGAGCGCAGATACC
AAATACTGTTCTTCTAGTGTAGCCGTAGTTAGCCCACCACTTCAAGAACT
CTGTAGCACCGCCTACATACCTCGCTCTGCTAATCCTGTTACCAGTGGCT
GCTGCCAGTGGCGATAAGTCGTGTCTTACCGGGTTGGACTCAAGACGATA
GTTACCGGATAAGGCGCAGCGGTCGGGCTGAACGGGGGGTTCGTGCACAC
AGCCCAGCTTGGAGCGAACGACCTACACCGAACTGAGATACCTACAGCGT
GAGCTATGAGAAAGCGCCACGCTTCCCGAAGGGAGAAAGGCGGACAGGTA
TCCGGTAAGCGGCAGGGTCGGAACAGGAGAGCGCACGAGGGAGCTTCCAG
GGGGAAACGCCTGGTATCTTTATAGTCCTGTCGGGTTTCGCCACCTCTGA
CTTGAGCGTCGATTTTTGTGATGCTCGTCAGGGGGGCGGAGCCTATGGAA
AAACGCCAGCAACGCGGCCTTTTTACGGTTCCTGGCCTTTTGCTGGCCTT
TTGCTCACATGTTCTTTCCTGCGTTATCCCCTGATTCTGTGGATAACCGT
ATTACCGCCTTTGAGTGAGCTGATACCGCTCGCCGCAGCCGAACGACCGA
GCGCAGCGAGTCAGTGAGCGAGGAAGCGGAAGGCGAGAGTAGGGAACTGC
CAGGCATCAAACTAAGCAGAAGGCCCCTGACGGATGGCCTTTTTGCGTTT
CTACAAACTCTTTCTGTGTTGTAAAACGACGGCCAGTCTTAAGCTCGGGC
CCCCTGGGCGGTTCTGATAACGAGTAATCGTTAATCCGCAAATAACGTAA
AAACCCGCTTCGGCGGGTTTTTTTATGGGGGGAGTTTAGGGAAAGAGCAT
TTGTCAGAATATTTAAGGGCGCCTGTCACTTTGCTTGATATATGAGAATT
ATTTAACCTTATAAATGAGAAAAAAGCAACGCACTTTAAATAAGATACGT
TGCTTTTTCGATTGATGAACACCTATAATTAAACTATTCATCTATTATTT
ATGATTTTTTGTATATACAATATTTCTAGTTTGTTAAAGAGAATTAAGAA
AATAAATCTCGAAAATAATAAAGGGAAAATCAGTTTTTGATATCAAAATT
ATACATGTCAACGATAATACAAAATATAATACAAACTATAAGATGTTATC
AGTATTTATTATCATTTAGAATAAATTTTGTGTCGCCCTTAATTGTGAGC
GGATAACAATTACGAGCTTCATGCACAGTGGCGTTGACATTGATTATTGA
CTAGTTATTAATAGTAATCAATTACGGGGTCATTAGTTCATAGCCCATAT
ATGGAGTTCCGCGTTACATAACTTACGGTAAATGGCCCGCCTGGCTGACC
GCCCAACGACCCCCGCCCATTGACGTCAATAATGACGTATGTTCCCATAG
TAACGCCAATAGGGACTTTCCATTGACGTCAATGGGTGGAGTATTTACGG
TAAACTGCCCACTTGGCAGTACATCAAGTGTATCATATGCCAAGTACGCC
CCCTATTGACGTCAATGACGGTAAATGGCCCGCCTGGCATTATGCCCAGT
ACATGACCTTATGGGACTTTCCTACTTGGCAGTACATCTACGTATTAGTC
ATCGCTATTACCATGGTGATGCGGTTTTGGCAGTACATCAATGGGCGTGG
ATAGCGGTTTGACTCACGGGGATTTCCAAGTCTCCACCCCATTGACGTCA
ATGGGAGTTTGTTTTGGCACCAAAATCAACGGGACTTTCCAAAATGTCGT
AACAACTCCGCCCCATTGACGCAAATGGGCGGTAGGCGTGTACGGTGGGA
GGTCTATATAAGCAGAGCTCTCTGGCTAACTAGAGAACCCACTGCTTACT
GGCTTATCGAAATTAATACGACTCACTATAGGGAAGCTTCTTGTTCTTTT
TGCAGAAGCTCAGAATAAACGCTCAACTTTGGCCTCGAGGCCACCATGGA
CTATAAGGACCACGACGGAGACTACAAGGATCATGATATTGATTACAAAG
ACGATGACGATAAGATGGCCCCAAAGAAGAAGCGGAAGGTCGGTATCCAC
GGAGTCCCAGCAGCCGTAGATTTGAGAACTTTGGGATATTCACAGCAGCA
GCAGGAAAAGATCAAGCCCAAAGTGAGGTCGACAGTCGCGCAGCATCACG
AAGCGCTGGTGGGTCATGGGTTTACACATGCCCACATCGTAGCCTTGTCG
CAGCACCCTGCAGCCCTTGGCACGGTCGCCGTCAAGTACCAGGACATGAT
TGCGGCGTTGCCGGAAGCCACACATGAGGCGATCGTCGGTGTGGGGAAAC
AGTGGAGCGGAGCCCGAGCGCTTGAGGCCCTGTTGACGGTCGCGGGAGAG
CTGAGAGGGCCTCCCCTTCAGCTGGACACGGGCCAGTTGCTGAAGATCGC
GAAGCGGGGAGGAGTCACGGCGGTCGAGGCGGTGCACGCGTGGCGCAATG
CGCTCACGGGAGCACCCCTCAACCTGACCCCAGACCAGGTTGTGGCCATC
GCCAGCAACATAGGTGGCAAGCAGGCCCTCGAAACCGTCCAGAGACTGTT
ACCGGTTCTCTGCCAGGACCACGGCCTGACCCCGGAACAGGTGGTTGCAA
TCGCGTCACACGATGGGGGAAAGCAGGCCCTAGAAACCGTTCAGCGACTC
CTGCCCGTCCTGTGCCAGGCCCACGGCCTGACCCCGGACCAGGTGGTTGC
AATCGCGTCACACGATGGGGGAAAGCAGGCCCTAGAAACCGTTCAGCGAC
TCCTGCCCGTCCTGTGCCAGGCCCACGGCCTGACCCCCGCCCAGGTTGTC
GCTATTGCTAGTAACGGCGGAGGCAAACAGGCGCTGGAAACAGTTCAGCG
CCTCTTGCCGGTCTTGTGTCAGGACCACGGCCTGACCCCAGACCAAGTTG
TCGCGATTGCAAGCAACAACGGAGGCAAACAAGCCTTAGAAACAGTCCAG
AGATTGTTGCCGGTGCTGTGCCAAGACCACGGCCTGACCCCAGAACAAGT
TGTCGCGATTGCAAGCAACAACGGAGGCAAACAAGCCTTAGAAACAGTCC
AGAGATTGTTGCCGGTGCTGTGCCAAGCCCACGGCCTGACCCCAGACCAG
GTTGTGGCCATCGCCAGCAACATAGGTGGCAAGCAGGCCCTCGAAACCGT
CCAGAGACTGTTACCGGTTCTCTGCCAGGCCCACGGCCTGACCCCAGCCC
AGGTTGTGGCCATCGCCAGCAACATAGGTGGCAAGCAGGCCCTCGAAACC
GTCCAGAGACTGTTACCGGTTCTCTGCCAGGACCACGGCCTGACCCCCGA
CCAGGTTGTCGCTATTGCTAGTAACGGCGGAGGCAAACAGGCGCTGGAAA
CAGTTCAGCGCCTCTTGCCGGTCTTGTGTCAGGACCACGGCCTGACCCCC
GAACAGGTTGTCGCTATTGCTAGTAACGGCGGAGGCAAACAGGCGCTGGA
AACAGTTCAGCGCCTCTTGCCGGTCTTGTGTCAGGCCCACGGCCTGACCC
CCGACCAGGTTGTCGCTATTGCTAGTAACGGCGGAGGCAAACAGGCGCTG
GAAACAGTTCAGCGCCTCTTGCCGGTCTTGTGTCAGGCCCACGGCCTGAC
CCCCGCCCAGGTTGTCGCTATTGCTAGTAACGGCGGAGGCAAACAGGCGC
TGGAAACAGTTCAGCGCCTCTTGCCGGTCTTGTGTCAGGACCACGGCCTG
ACCCCAGACCAAGTTGTCGCGATTGCAAGCAACAACGGAGGCAAACAAGC
CTTAGAAACAGTCCAGAGATTGTTGCCGGTGCTGTGCCAAGACCACGGCC
TGACCCCCGAACAGGTTGTCGCTATTGCTAGTAACGGCGGAGGCAAACAG
GCGCTGGAAACAGTTCAGCGCCTCTTGCCGGTCTTGTGTCAGGCCCACGG
CCTGACCCCGGACCAGGTGGTTGCAATCGCGTCACACGATGGGGGAAAGC
AGGCCCTAGAAACCGTTCAGCGACTCCTGCCCGTCCTGTGCCAGGCCCAC
GGCCTGACCCCCGCCCAGGTTGTCGCTATTGCTAGTAACGGCGGAGGCAA
ACAGGCGCTGGAAACAGTTCAGCGCCTCTTGCCGGTCTTGTGTCAGGACC
ACGGCCTGACGCCTGAGCAGGTAGTGGCTATTGCATCCAACGGAGGGGGC
AGACCCGCACTGGAGTCAATCGTGGCCCAGCTTTCGAGGCCGGACCCCGC
GCTGGCCGCACTCACTAATGATCATCTTGTAGCGCTGGCCTGCCTCGGCG
GACGACCCGCCTTGGATGCGGTGA.
SEQ ID NO: 2
AGAAGGGGCTCCCGCACGCGCCTGCATTGATTAAGCGGACCAACAGAAGG
ATCCCCGAGAGGACATCACATCGAGTGGCAGGTTCCCAACTCGTGAAGAG
TGAACTTGAGGAGAAAAAGTCGGAGCTGCGGCACAAATTGAAATACGTAC
CGCATGAATACATCGAACTTATCGAAATTGCTAGGAACTCGACTCAAGAC
AGAATCCTTGAGATGAAGGTAATGGAGTTCTTTATGAAGGTTTATGGATA
CCGAGGGAAGCATCTCGGTGGATCACGAAAACCCGACGGAGCAATCTATA
CGGTGGGGAGCCCGATTGATTACGGAGTGATCGTCGACACGAAAGCCTAC
AGCGGTGGGTACAATCTTCCCATCGGGCAGGCAGATGAGATGCAACGTTA
TGTCGAAGAAAATCAGACCAGGAACAAACACATCAATCCAAATGAGTGGT
GGAAAGTGTATCCTTCATCAGTGACCGAGTTTAAGTTTTTGTTTGTCTCT
GGGCATTTCAAAGGCAACTATAAGGCCCAGCTCACACGGTTGAATCACAT
TACGAACTGCAATGGTGCGGTTTTGTCCGTAGAGGAACTGCTCATTGGTG
GAGAAATGATCAAAGCGGGAACTCTGACACTGGAAGAAGTCAGACGCAAG
TTTAACAATGGCGAGATCAATTTCCGCTCATAAAAAATCAGCCTCGACTG
TGCCTTCTAGTTGCCAGCCATCTGTTGTTTGCCCCTCCCCCGTGCCTTCC
TTGACCCTGGAAGGTGCCACTCCCACTGTCCTTTCCTAATAAAATGAGGA
AATTGCATCACAACACTCAACCCTATCTCGGTCTATTCTTTTGATTTATA
AGGGATTTTGCCGATTTCGGCCTATTGGTTAAAAAATGAGCTGATTTAAC
AAAAATTTAACGCGAATTAATTCTGTGGAATGTGTGTCAGTTAGGGTGTG
GAAAGTCCCCAGGCTCCCCAGCAGGCAGAAGTATGCAAAGCATGCATCTC
AATTAGTCAGCAACCAGGTGTGGAAAGTCCCCAGGCTCCCCAGCAGGCAG
AAGTATGCAAAGCATGCATCTCAATTAGTCAGCAACCATAGTCCCGCCCC
TAACTCCGCCCATCCCGCCCCTAACTCCGCCCAGTTCCGCCCATTCTCCG
CCCCATGGCTGACTAATTTTTTTTATTTATGCAGAGGCCGAGGCCGCCTC
TGCCTCTGAGCTATTCCAGAAGTAGTGAGGAGGCTTTTTTGGAGGCCTAG
GCTTTTGCAAAAAGCTCCCGGGAGCTTGTATATCCATTTTCGGATCTGAT
CAGCACGTGATGAAAAAGCCTGAACTCACCGCGACGTCTGTCGAGAAGTT
TCTGATCGAAAAGTTCGACAGCGTTTCCGACCTGATGCAGCTCTCGGAGG
GCGAAGAATCTCGTGCTTTCAGCTTCGATGTAGGAGGGCGTGGATATGTC
CTGCGGGTAAATAGCTGCGCCGATGGTTTCTACAAAGATCGTTATGTTTA
TCGGCACTTTGCATCGGCCGCGCTCCCGATTCCGGAAGTGCTTGACATTG
GGGAATTCAGCGAGAGCCTGACCTATTGCATCTCCCGCCGTGCACAGGGT
GTCACGTTGCAAGACCTGCCTGAAACCGAACTGCCCGCTGTTCTGCAGCC
GGTCGCGGAGGCCATGGATGCGATCGCTGCGGCCGATCTTAGCCAGACGA
GCGGGTTCGGCCCATTCGGACCGCAAGGAATCGGTCAATACACTACATGG
CGTGATTTCATATGCGCGATTGCTGATCCCCATGTGTATCACTGGCAAAC
TGTGATGGACGACACCGTCAGTGCGTCCGTCGCGCAGGCTCTCGATGAGC
TGATGCTTTGGGCCGAGGACTGCCCCGAAGTCCGGCACCTCGTGCACGCG
GATTTCGGCTCCAACAATGTCCTGACGGACAATGGCCGCATAACAGCGGT
CATTGACTGGAGCGAGGCGATGTTCGGGGATTCCCAATACGAGGTCGCCA
ACATCTTCTTCTGGAGGCCGTGGTTGGCTTGTATGGAGCAGCAGACGCGC
TACTTCGAGCGGAGGCATCCGGAGCTTGCAGGATCGCCGCGGCTCCGGGC
GTATATGCTCCGCATTGGTCTTGACCAACTCTATCAGAGCTTGGTTGACG
GCAATTTCGATGATGCAGCTTGGGCGCAGGGTCGATGCGACGCAATCGTC
CGATCCGGAGCCGGGACTGTCGGGCGTACACAAATCGCCCGCAGAAGCGC
GGCCGTCTGGACCGATGGCTGTGTAGAAGTACTCGCCGATAGTGGAAACC
GACGCCCCAGCACTCGTCCGAGGGCAAAGGAATAGCACGTGCTACGAGAT
TTCGATTCCACCGCCGCCTTCTATGAAAGGTTGGGCTTCGGAATCGTTTT
CCGGGACGCCGGCTGGATGATCCTCCAGCGCGGGGATCTCATGCTGGAGT
TCTTCGCCCACCCCAACTTGTTTATTGCAGCTTATAATGGTTACAAATAA
AGCAATAGCATCACAAATTTCACAAATAAAGCATTTTTTTCACTGCATTC
TAGTTGTGGTTTGTCCAAACTCATCAATGTATCTTATCATGTCTGTATAC
CGTCGACCTCTAGCTAGAGCTTGGCGTAATCATGGTCATTACCAATGCTT
AATCAGTGAGGCACCTATCTCAGCGATCTGTCTATTTCGTTCATCCATAG
TTGCCTGACTCCCCGTCGTGTAGATAACTACGATACGGGAGGGCTTACCA
TCTGGCCCCAGCGCTGCGATGATACCGCGAGAACCACGCTCACCGGCTCC
GGATTTATCAGCAATAAACCAGCCAGCCGGAAGGGCCGAGCGCAGAAGTG
GTCCTGCAACTTTATCCGCCTCCATCCAGTCTATTAATTGTTGCCGGGAA
GCTAGAGTAAGTAGTTCGCCAGTTAATAGTTTGCGCAACGTTGTTGCCAT
CGCTACAGGCATCGTGGTGTCACGCTCGTCGTTTGGTATGGCTTCATTCA
GCTCCGGTTCCCAACGATCAAGGCGAGTTACATGATCCCCCATGTTGTGC
AAAAAAGCGGTTAGCTCCTTCGGTCCTCCGATCGTTGTCAGAAGTAAGTT
GGCCGCAGTGTTATCACTCATGGTTATGGCAGCACTGCATAATTCTCTTA
CTGTCATGCCATCCGTAAGATGCTTTTCTGTGACTGGTGAGTACTCAACC
AAGTCATTCTGAGAATAGTGTATGCGGCGACCGAGTTGCTCTTGCCCGGC
GTCAATACGGGATAATACCGCGCCACATAGCAGAACTTTAAAAGTGCTCA
TCATTGGAAAACGTTCTTCGGGGCGAAAACTCTCAAGGATCTTACCGCTG
TTGAGATCCAGTTCGATGTAACCCACTCGTGCACCCAACTGATCTTCAGC
ATCTTTTACTTTCACCAGCGTTTCTGGGTGAGCAAAAACAGGAAGGCAAA
ATGCCGCAAAAAAGGGAATAAGGGCGACACGGAAATGTTGAATACTCATA
TTCTTCCTTTTTCAATATTATTGAAGCATTTATCAGGGTTATTGTCTCAT
GAGCGGATACATATTTGAATGTATTTAGAAAAATAAACAAATAGGGGTCA
GTGTTACAACCAATTAACCAATTCTGAACATTATCGCGAGCCCATTTATA
CCTGAATATGGCTCATAACACCCCTTGCTCATGACCAAAATCCCTTAACG
TGAGTTACGCGCGCGTCGTTCCACTGAGCGTCAGACCCCGTAGAAAAGAT
CAAAGGATCTTCTTGAGATCCTTTTTTTCTGCGCGTAATCTGCTGCTTGC
AAACAAAAAAACCACCGCTACCAGCGGTGGTTTGTTTGCCGGATCAAGAG
CTACCAACTCTTTTTCCGAAGGTAACTGGCTTCAGCAGAGCGCAGATACC
AAATACTGTTCTTCTAGTGTAGCCGTAGTTAGCCCACCACTTCAAGAACT
CTGTAGCACCGCCTACATACCTCGCTCTGCTAATCCTGTTACCAGTGGCT
GCTGCCAGTGGCGATAAGTCGTGTCTTACCGGGTTGGACTCAAGACGATA
GTTACCGGATAAGGCGCAGCGGTCGGGCTGAACGGGGGGTTCGTGCACAC
AGCCCAGCTTGGAGCGAACGACCTACACCGAACTGAGATACCTACAGCGT
GAGCTATGAGAAAGCGCCACGCTTCCCGAAGGGAGAAAGGCGGACAGGTA
TCCGGTAAGCGGCAGGGTCGGAACAGGAGAGCGCACGAGGGAGCTTCCAG
GGGGAAACGCCTGGTATCTTTATAGTCCTGTCGGGTTTCGCCACCTCTGA
CTTGAGCGTCGATTTTTGTGATGcTCGTCAGGGGGGCGGAGCCTATGGAA
AAACGCCAGCAACGCGGCCTTTTTACGGTTCCTGGCCTTTTGCTGGCCTT
TTGCTCACATGTTCTTTCCTGCGTTATCCCCTGATTCTGTGGATAACCGT
ATTACCGCCTTTGAGTGAGCTGATACCGCTCGCCGCAGCCGAACGACCGA
GCGCAGCGAGTCAGTGAGCGAGGAAGCGGAAGGCGAGAGTAGGGAACTGC
CAGGCATCAAACTAAGCAGAAGGCCCCTGACGGATGGCCTTTTTGCGTTT
CTACAAACTCTTTCTGTGTTGTAAAACGACGGCCAGTCTTAAGCTCGGGC
CCCCTGGGCGGTTCTGATAACGAGTAATCGTTAATCCGCAAATAACGTAA
AAACCCGCTTCGGCGGGTTTTTTTATGGGGGGAGTTTAGGGAAAGAGCAT
TTGTCAGAATATTTAAGGGCGCCTGTCACTTTGCTTGATATATGAGAATT
ATTTAACCTTATAAATGAGAAAAAAGCAACGCACTTTAAATAAGATACGT
TGCTTTTTCGATTGATGAACACCTATAATTAAACTATTCATCTATTATTT
ATGATTTTTTGTATATACAATATTTCTAGTTTGTTAAAGAGAATTAAGAA
AATAAATCTCGAAAATAATAAAGGGAAAATCAGTTTTTGATATCAAAATT
ATACATGTCAACGATAATACAAAATATAATACAAACTATAAGATGTTATC
AGTATTTATTATCATTTAGAATAAATTTTGTGTCGCCCTTAATTGTGAGC
GGATAACAATTACGAGCTTCATGCACAGTGGCGTTGACATTGATTATTGA
CTAGTTATTAATAGTAATCAATTACGGGGTCATTAGTTCATAGCCCATAT
ATGGAGTTCCGCGTTACATAACTTACGGTAAATGGCCCGCCTGGCTGACC
GCCCAACGACCCCCGCCCATTGACGTCAATAATGACGTATGTTCCCATAG
TAACGCCAATAGGGACTTTCCATTGACGTCAATGGGTGGAGTATTTACGG
TAAACTGCCCACTTGGCAGTACATCAAGTGTATCATATGCCAAGTACGCC
CCCTATTGACGTCAATGACGGTAAATGGCCCGCCTGGCATTATGCCCAGT
ACATGACCTTATGGGACTTTCCTACTTGGCAGTACATCTACGTATTAGTC
ATCGCTATTACCATGGTGATGCGGTTTTGGCAGTACATCAATGGGCGTGG
ATAGCGGTTTGACTCACGGGGATTTCCAAGTCTCCACCCCATTGACGTCA
ATGGGAGTTTGTTTTGGCACCAAAATCAACGGGACTTTCCAAAATGTCGT
AACAACTCCGCCCCATTGACGCAAATGGGCGGTAGGCGTGTACGGTGGGA
GGTCTATATAAGCAGAGCTCTCTGGCTAACTAGAGAACCCACTGCTTACT
GGCTTATCGAAATTAATACGACTCACTATAGGGAAGCTTCTTGTTCTTTT
TGCAGAAGCTCAGAATAAACGCTCAACTTTGGCCTCGAGGCCACCATGGA
CTATAAGGACCACGACGGAGACTACAAGGATCATGATATTGATTACAAAG
ACGATGACGATAAGATGGCCCCAAAGAAGAAGCGGAAGGTCGGTATCCAC
GGAGTCCCAGCAGCCGTAGATTTGAGAACTTTGGGATATTCACAGCAGCA
GCAGGAAAAGATCAAGCCCAAAGTGAGGTCGACAGTCGCGCAGCATCACG
AAGCGCTGGTGGGTCATGGGTTTACACATGCCCACATCGTAGCCTTGTCG
CAGCACCCTGCAGCCCTTGGCACGGTCGCCGTCAAGTACCAGGACATGAT
TGCGGCGTTGCCGGAAGCCACACATGAGGCGATCGTCGGTGTGGGGAAAC
AGTGGAGCGGAGCCCGAGCGCTTGAGGCCCTGTTGACGGTCGCGGGAGAG
CTGAGAGGGCCTCCCCTTCAGCTGGACACGGGCCAGTTGCTGAAGATCGC
GAAGCGGGGAGGAGTCACGGCGGTCGAGGCGGTGCACGCGTGGCACAATG
CGCTCACGGGAGCACCCCTCAACCTGACCCCAGACCAGGTTGTGGCCATC
GCCAGCAACATAGGTGGCAAGCAGGCCCTCGAAACCGTCCAGAGACTGTT
ACCGGTTCTCTGCCAGGACCACGGCCTGACCCCCGAACAGGTTGTCGCTA
TTGCTAGTAACGGCGGAGGCAAACAGGCGCTGGAAACAGTTCAGCGCCTC
TTGCCGGTCTTGTGTCAGGCCCACGGCCTGACCCCCGACCAGGTTGTCGC
TATTGCTAGTAACGGCGGAGGCAAACAGGCGCTGGAAACAGTTCAGCGCC
TCTTGCCGGTCTTGTGTCAGGCCCACGGCCTGACCCCGGCCCAGGTGGTT
GCAATCGCGTCACACGATGGGGGAAAGCAGGCCCTAGAAACCGTTCAGCG
ACTCCTGCCCGTCCTGTGCCAGGACCACGGCCTGACCCCAGACCAGGTTG
TGGCCATCGCCAGCAACATAGGTGGCAAGCAGGCCCTCGAAACCGTCCAG
AGACTGTTACCGGTTCTCTGCCAGGACCACGGCCTGACCCCGGAACAGGT
GGTTGCAATCGCGTCACACGATGGGGGAAAGCAGGCCCTAGAAACCGTTC
AGCGACTCCTGCCCGTCCTGTGCCAGGCCCACGGCCTGACCCCGGACCAG
GTGGTTGCAATCGCGTCACACGATGGGGGAAAGCAGGCCCTAGAAACCGT
TCAGCGACTCCTGCCCGTCCTGTGCCAGGCCCACGGCCTGACCCCAGCCC
AGGTTGTGGCCATCGCCAGCAACATAGGTGGCAAGCAGGCCCTCGAAACC
GTCCAGAGACTGTTACCGGTTCTCTGCCAGGACCACGGCCTGACCCCAGA
CCAGGTTGTGGCCATCGCCAGCAACATAGGTGGCAAGCAGGCCCTCGAAA
CCGTCCAGAGACTGTTACCGGTTCTCTGCCAGGACCACGGCCTGACCCCG
GAACAGGTGGTTGCAATCGCGTCACACGATGGGGGAAAGCAGGCCCTAGA
AACCGTTCAGCGACTCCTGCCCGTCCTGTGCCAGGCCCACGGCCTGACCC
CGGACCAGGTGGTTGCAATCGCGTCACACGATGGGGGAAAGCAGGCCCTA
GAAACCGTTCAGCGACTCCTGCCCGTCCTGTGCCAGGCCCACGGCCTGAC
CCCAGCCCAGGTTGTGGCCATCGCCAGCAACATAGGTGGCAAGCAGGCCC
TCGAAACCGTCCAGAGACTGTTACCGGTTCTCTGCCAGGACCACGGCCTG
ACCCCGGACCAGGTGGTTGCAATCGCGTCACACGATGGGGGAAAGCAGGC
CCTAGAAACCGTTCAGCGACTCCTGCCCGTCCTGTGCCAGGACCACGGCC
TGACCCCAGAACAGGTTGTGGCCATCGCCAGCAACATAGGTGGCAAGCAG
GCCCTCGAAACCGTCCAGAGACTGTTACCGGTTCTCTGCCAGGCCCACGG
CCTGACCCCGGACCAGGTGGTTGCAATCGCGTCACACGATGGGGGAAAGC
AGGCCCTAGAAACCGTTCAGCGACTCCTGCCCGTCCTGTGCCAGGCCCAC
GGCCTGACCCCGGCCCAGGTGGTTGCAATCGCGTCACACGATGGGGGAAA
GCAGGCCCTAGAAACCGTTCAGCGACTCCTGCCCGTCCTGTGCCAGGACC
ACGGCCTGACGCCTGAGCAGGTAGTGGCTATTGCATCCAACATAGGGGGC
AGACCCGCACTGGAGTCAATCGTGGCCCAGCTTTCGAGGCCGGACCCCGC
GCTGGCCGCACTCACTAATGATCATCTTGTAGCGCTGGCCTGCCTCGGCG
GACGACCCGCCTTGGATGCGGTGA.

In Vitro Maturation and In Vitro Fertilization

The TALEN injected ova were incubated for 24 hours or more in Porcine Oocyte Medium (Research Institute for the Functional Peptides, IFP1010P) containing inactivated FBS (final concentration: 5%), hFSH (Fuji Pharma, final concentration: 0.15 IU/mL), and hCG (ASKA Pharmaceutical, final concentration: 10 IU/mL). Then, the ova matured to MI (Metaphase I) or more were collected. Semen used for fertilization was collected from wild-type male marmosets, and in vitro fertilization (IVF) was carried out with use of TYH medium (LSI medience, DR01031). The next day (approximately 16 hours after the start of IVF), IVF was released. It was determined whether the fertilization was succeeded or not in accordance with the presence or absence of a prokaryotic cell. The fertilized ova thus obtained were collected.

Embryo Transfer and Pregnancy Management

The fertilized ova were subjected to early-stage in vitro incubation in Sequencial Cleav (Origio, 83040010A). Two or three days after the start of the incubation, the embryos grown to a 4-cell stage or higher were collected. The embryos thus collected were subjected to later-stage in vitro incubation in a medium that was Sequencial Blast (Origio, 83060010A) containing inactivated FBS (final concentration: 10%) and L-glutamine (final concentration: 2 mM). One week after the injection of TALEN, the embryos were transferred into the uteri of surrogate mothers through a noninvasive catheter operation. After the transfer of the embryos, the surrogate mothers were measured for a P4-value in the blood and undergone uterus observation by ultrasonic diagnostic equipment so as to determine pregnancy. Individuals confirmed to be pregnant undergone a periodical examination once a week so as to observe a fetal heart rate, and the presence or absence of a morphological abnormality, etc. A method for obtaining newborns was based on natural delivery. If natural delivery was judged to be difficult from the state(s) of the surrogate mother and/or fetus, a caesarean section was performed to obtain a newborn.

Genomic DNA Analysis

Genomic DNA was extracted, with use of QIAamp DNA

Micro Kit (Qiagen, 56304), from hair of a newborn or cord blood obtained at the caesarean section. PCR for analyzing the PSEN1 gene was carried out with use of KOD plus neo (TOYOBO, 401) and two primers, Psen_1_Ex9_up1 (ACCCGCGACTCCCTATTATT: SEQ ID NO: 3) and Psen_1_Ex9_dn1 (TGCCTTGACTGTATTGTTGG: SEQ ID NO: 4). The reaction was carried out under the following reaction condition: After heating at 94° C. for 2 minutes, 35 cycles of 98° C. for 10 seconds, 60° C. for 10 seconds, and 68° C. for 30 seconds were carried out. After the reaction, some of the resultants were subjected to electrophoresis on an agarose gel to check for amplification. Some of the amplification products were subjected to surveyor assay, with which genetic modification can be visually detected. The remaining ones were cloned to a sequence vector with use of Zero Blunt PCR Cloning Kit (Thermo fisher scientific, K275040), and the plasmids were introduced into Escherichia coli (strain DH5a) for transformation. From the resulting subclones, plasmids were collected and subjected to sequence analysis.

cDNA Analysis

Total RNA was extracted, with use of Nucleospin RNA plus XS (Takara, U0990B), from hair of a newborn or cord blood obtained at the caesarean section. Then, cDNA was synthesized with use of ReverTra Ace-α-(TOYOBO, FSK-101). PCR for checking for the deficiency of exon 9 in the PSEN1 gene was carried out with use of KOD plus neo and a set of two primers designed on an exon in the vicinity of the deficient site of exon 9. The set of two primers was either of (i) Pn1_on_Ex7_up1 (TACCTCCCTGAATGGACTGC: SEQ ID NO: 5) and Pn1_on_Ex11_dn2 (TGGTTGTGTTCCAGTCTCCA: SEQ ID NO: 6) and (ii) Pn1_on_Ex8_up1 (GGTCCACTTCGTATGCTGGT: SEQ ID NO: 7) and Pn1_on_Ex11_dn1 (GGCTGTTGCTGAGGCTTTAC: SEQ ID NO: 8). The reaction was carried out under the following reaction condition: After heating at 94° C. for 2 minutes, 35 cycles of 98° C. for 10 seconds, 60° C. for 10 seconds, and 68° C. for 30 seconds were carried out. The samples undergone the reaction were subjected to electrophoresis on a 1.5% agarose gel. In the amplified products, band shifting was found to estimate the deficiency of exon 9. Some of the amplified products were cloned to a sequence vector with use of Zero Blunt PCR Cloning Kit, and the plasmids were introduced into Escherichia coli (strain DH5a) for transformation. From the resulting subclones, plasmids were collected and subjected to sequence analysis.

Primary Culture

A small amount of tissues were excised from ear lobes of newborns, and were cut into small pieces. The tissue pieces were put on a cell culture dish, and were cultured at 37° C. in a 5% CO₂ environment with use of D-MEM (Thermo fisher scientific, 10566016) containing 10% inactivated FBS.

### Example 1

TALEN mRNAs targeting the 3′-splice site of exon 9 in the marmoset PSEN1 gene were introduced into the nuclei of marmoset fertilized ova. Then, subcloning was carried out, followed by surveyor assay and sequencing assay. As shown in a of FIG. 2, the deficiency of the target site including the 3′-splice site sequence was found in two out of three fertilized ova, as expected. After the introduction of TALEN into the marmoset fertilized ova, RT-PCR and cDNA sequencing were carried out. Consequently, exclusion of exon 9 in the PSEN1 mRNA was verified. As shown in b of FIG. 2, after the introduction of TALEN, an RNA was extracted from a 4-cell-stage fertilized ovum or a single blastomere. As shown in c of FIG. 2, complete exclusion of exon 9 was found in two 4-cell-stage fertilized ova and two single blastomeres. There was no wild-type sequence in the fertilized ova and single blastomeres in which the 3′-splice site was deficient. From this, it is considered that the 3′-splice site was made deficient in a biallelic manner. PS1 protein is a catalytic subunit for a γ-secretase complex having substrates that are type 1 transmembrane proteins including amyloid precursor protein (APP) and Notch. A region encoded by exon 9 exits in a site for endoproteolysis, and cleavage at this site is necessary for maturation of γ-secretase. Thus, exclusion of exon 9 leads to loss of the function of γ-secretase. The homozygous deletion of exon 9 in marmoset PSEN1 causes embryonic lethality due to destruction of Notch signaling.

### Example 2

On the basis of the results obtained from the fertilized ova, an attempt was made to introduce TALEN mRNAs into ova in the above-described manner and to fertilize the ova. After fertilization, the fertilized ova were developed to the morula stage, and were then transferred to surrogate mothers. The introduction of TALEN did not cause any distinct adverse reaction in pregnancy or following development. In this manner, F0 marmosets (PSEN1-ΔE9) carrying a 6-bp deletion including the 3′-splice site in a heterozygous state could be obtained (a of FIG. 3 shows a photograph of a neonate). Genomic DNA extracted from the cord blood was subjected to surveyor assay. A result thereof is shown in b of FIG. 3. In b of FIG. 3, two left columns indicate results of the surveyor assay (Lane 1: a mixture of the genomic DNA of the neonate shown in a of FIG. 3 and a wild-type control blood (CB) sample, Lane 2: only the neonate genomic DNA) carried out by using, as a template, the neonate genomic DNA (the genomic DNA extracted from the cord blood of the neonate shown in a of FIG. 3). In b of FIG. 3, two right columns indicate controls for the experiment (“+” is a positive control, and “−” is a negative control). c of FIG. 3 shows results of PCR carried out using RNAs extracted from hair roots of the obtained mutant marmoset and the wild-type marmoset. In c of FIG. 3, two right columns indicate negative PCR data obtained from reactions of the same RNA samples taken place without RT. As indicated in c of FIG. 3, the PCR product with exclusion of exon 9 appeared as a 315 bp band, whereas the wild-type appeared as a 402 bp band. Note that the results obtained from the analyzed fertilized ova each indicated only a single band. From this, it is considered that a heterozygous mutation did not take place.

### Example 3

ELISA (Wako, 294-62501, 290-62601) was used to quantify Aβ in culture media of primary skin fibroblasts of a wild-type marmoset and PSEN1-ΔE9 (I774gmF) marmoset. A result thereof is shown in FIG. 4. In the fibroblasts of the PSEN1-ΔE9 marmoset, a decrease of Aβ₄₀, an increase of Aβ₄₂, and an increase of Aβ₄₂/Aβ₄₀ ratio were found. This shows that PSEN1-ΔE9 mutation has pathogenicity (i.e., PSEN1-ΔE9 mutation causes a disease).

Genomic DNA analysis and cDNA analysis indicated that, in two individuals with a mutation in the PSEN1 gene, normal PSEN1 and exon 9-deficient PSEN1 appeared at a ratio of approximately 1:1. This strongly suggests that heterozygous state of PSEN1-ΔE9, which is found in human patients, could be replicated. In addition, according to the result of Aβ quantification in the fibroblast culture media, replication of an abnormality in Aβ production profile was found. From this, it is considered that the produced PSEN1-ΔE9 marmosets replicated a disease state of AD. The method, adopted in the Examples, for introducing a mutation in the 3′-splice site to induce exon skipping can be carried out by inducing DNA double strand break at a single site on a genomic DNA, and thus is considered to be more efficient and useful than a generally-conducted method for deleting an exon by cleaving both ends of the exon by genome editing and a knock-in method. Such a method for introducing, by genome editing, a mutation into a sequence related to splicing to produce a disease model that replicates a state of a disease (Alzheimer's disease) is considered to be useful. Furthermore, in a case where, as in the present invention, a targeted gene mutation can cause embryonic lethality if the gene mutation takes place in a homozygous status, a method for carrying out genome editing on ova and fertilizing the ova is considered to be useful for avoiding embryonic lethality.

### Example 4

In the same manner as that in Example 2, two PSEN1-ΔE9 neonate individuals (I943gmM and I949gmM) were further obtained (a of FIG. 5). For three individuals including I774gmF in addition to the two PSEN1-ΔE9 neonate individuals, surveyor assay was carried out on genomic DNAs extracted from cord blood. A result thereof is shown in b of FIG. 5. The meanings of the letters indicated in a lower part of b of FIG. 5 are as follows. “CB” denotes a genome extracted from cord blood of a neonate. “HR” denotes a genome extracted from hair roots of the neonate. “M” denotes a marker. “+N” indicates addition of Normal Genome. “+” and “−” indicate reagent controls. As indicated in c of FIG. 5, I774gmF and I943gmM had the PSEN1 gene carrying a 6-bp deletion including the 3′-splice site in a heterozygous state, whereas I949gmM had the PSEN1 gene carrying a 9-bp deletion including the 3′-splice site in a heterozygous state.

Western blot analysis was carried out against PS1 protein in membrane fractions obtained from the fibroblasts of the wild-type (I774gmF) and PSEN1-ΔE9 (I943gmM and I949gmM) marmosets. A result thereof is shown in a of FIG. 6. A left panel shows a result of the analysis involving use of an antibody to an N-terminal fragment (NTF) of the PS1 protein. A right panel shows a result of the analysis involving use of an antibody to a C-terminal fragment (CTF) of the PS1 protein. The arrowheads indicate NTF and CTF produced by endoproteolysis of PS1, whereas the arrows indicate a full-length PS1 protein. Sodium-potassium ATPase was used as a loading control of the membrane fraction.

In the culture media of the primary skin fibroblasts, Aβ was quantified by sandwich ELISA (Wako, 294-62501, 290-62601). A result thereof is shown in c of FIG. 6. c of FIG. 6 indicates an average value±a standard error (WT: n=15, ΔE9: n=9). In c of FIG. 6, “**P≤0.01” and “****≤P0.0001” each indicate the presence of a significant difference as a result of Student's two-tailed t-test. Similarly to Example 3, a decrease of AΔ₄₀, an increase of Aβ₄₂, and an increase of Aβ₄₂/Aβ₄₀ ratio were found. The Aβ₄₃ level was less than a detection limit, and therefore is not illustrated.

### INDUSTRIAL APPLICABILITY

A non-human primate AD model in accordance with an aspect of the present invention is very useful for development of a method for diagnosis and therapy of Alzheimer's disease.

### REFERENCES

1. Saito, T. et al. Single App knock-in mouse models of Alzheimer's disease. Nat Neurosci 17, 661-663, doi:10.1038/nn.3697 (2014).
2. Sasaguri, H. et al. APP mouse models for Alzheimer's disease preclinical studies. EMBO J 36, 2473-2487, doi:10.15252/embj.201797397 (2017).
3. Sasaki, E. et al. Generation of transgenic non-human primates with germline transmission. Nature 459, 523-527, doi:10.1038/nature08090 (2009).
4. Izpisua Belmonte, J. C. et al. Brains, genes, and primates. Neuron 86, 617-631, doi:10.1016/j.neuron.2015.03.021 (2015).
5. Miller, C. T. et al. Marmosets: A Neuroscientific Model of Human Social Behavior. Neuron 90, 219-233, doi:10.1016/j.neuron.2016.03.018 (2016).
6. Crook, R. et al. A variant of Alzheimer's disease with spastic paraparesis and unusual plaques due to deletion of exon 9 of presenilin 1. Nature medicine 4, 452-455 (1998).
7. Prihar, G. et al. Alzheimer disease PS-1 exon 9 deletion defined. Nature medicine 5, 1090, doi:10.1038/13383 (1999).
8. Smith, M. J. et al. Variable phenotype of Alzheimer's disease with spastic paraparesis. Annals of neurology 49, 125-129 (2001).
9. Le Guennec, K. et al. Deletion of exons 9 and 10 of the Presenilin 1 gene in a patient with Early-onset Alzheimer Disease generates longer amyloid seeds. Neurobiology of disease 104, 97-103, doi:10.1016/j.nbd.2017.04.020 (2017).
10. Blauwendraat, C. et al. Pilot whole-exome sequencing of a German early-onset Alzheimer's disease cohort reveals a substantial frequency of PSEN2 variants. Neurobiology of aging 37, 208.e211-208.e217, doi:10.1016/j.neurobiolaging.2015.09.016 (2016).
11. Hutton, M. et al. Complete analysis of the presenilin 1 gene in early onset Alzheimer's disease. Neuroreport 7, 801-805 (1996).
12. Kwok, J. B. et al. Two novel (M233T and R278T) presenilin-1 mutations in early-onset Alzheimer's disease pedigrees and preliminary evidence for association of presenilin-1 mutations with a novel phenotype. Neuroreport 8, 1537-1542 (1997).
13. Brooks, W. S. et al. Alzheimer's disease with spastic paraparesis and ‘cotton wool’ plaques: two pedigrees with PS-1 exon 9 deletions. Brain: a journal of neurology 126, 783-791 (2003).
14. Sato, K. et al. Generation of a Nonhuman Primate Model of Severe Combined Immunodeficiency Using Highly Efficient Genome Editing. Cell stem cell 19, 127-138, doi:10.1016/j.stem.2016.06.003 (2016).
15. Tomita, T. & Iwatsubo, T. Structural biology of presenilins and signal peptide peptidases. The Journal of biological chemistry 288, 14673-14680, doi:10.1074/jbc.R113.463281 (2013).
16. Shen, J. et al. Skeletal and CNS defects in Presenilin-1-deficient mice. Cell 89, 629-639 (1997).
17. Kumar-Singh, S. et al. Mean age-of-onset of familial Alzheimer disease caused by presenilin mutations correlates with both increased A beta 42 and decreased A beta 40. Human Mutation 27, 686-695, doi:10.1002/humu.20336 (2006).
18. Sakuma, T. et al. Repeating pattern of non-RVD variations in DNA-binding modules enhances TALEN activity. Scientific reports 3, 3379, doi:10.1038/srep03379 (2013).
19. Geula, C., Nagykery, N. & Wu, C. K. Amyloid-beta deposits in the cerebral cortex of the aged common marmoset (Callithrix jacchus): incidence and chemical composition. Acta neuropathologica 103, 48-58 (2002).
20. Ridley, R. M., Baker, H. F., Windle, C. P. & Cummings, R. M. Very long term studies of the seeding of beta-amyloidosis in primates. Journal of neural transmission (Vienna, Austria: 1996) 113, 1243-1251, doi:10.1007/s00702-005-0385-2 (2006).
21. Rodriguez-Callejas, J. D., Fuchs, E. & Perez-Cruz, C. Evidence of Tau Hyperphosphorylation and Dystrophic Microglia in the Common Marmoset. Frontiers in aging neuroscience 8, 315, doi:10.3389/fnagi.2016.00315 (2016).
22. t Hart, B. A. & Massacesi, L. Clinical, pathological, and immunologic aspects of the multiple sclerosis model in common marmosets (Callithrix jacchus). Journal of neuropathology and experimental neurology 68, 341-355, doi:10.1097/NEN.0b013e31819f1d24 (2009).
23. Tardif, S. D., Mansfield, K. G., Ratnam, R., Ross, C. N. & Ziegler, T. E. The marmoset as a model of aging and age-related diseases. ILAR journal 52, 54-65 (2011).
24. Crofts, H. S. et al. Investigation of the sleep electrocorticogram of the common marmoset (Callithrix jacchus) using radiotelemetry. Clinical neurophysiology: official journal of the International Federation of Clinical Neurophysiology 112, 2265-2273 (2001).
25. Hikishima, K. et al. Atlas of the developing brain of the marmoset monkey constructed using magnetic resonance histology. Neuroscience 230, 102-113, doi:10.1016/j.neuroscience.2012.09.053 (2013).
26. Senoo, A., Tokuno, H. & Watson, C. Mini-atlas of the marmoset brain. Neuroscience research 93, 128-135, doi:10.1016/j.neures.2014.12.014 (2015).
27. Hashikawa, T., Nakatomi, R. & Iriki, A. Current models of the marmoset brain. Neuroscience research 93, 116-127, doi:10.1016/j.neures.2015.01.009 (2015).

1. A non-human primate model animal of Alzheimer's disease, comprising:
the PSEN1 gene in which a site including at least a 5′- or 3′-splice site of exon 9 and being related to splicing of exon 9 is deficient.

2. The non-human primate model animal of Alzheimer's disease as set forth in claim 1, wherein
the non-human primate is a marmoset.

3. A method for producing a non-human primate model animal of Alzheimer's disease, the method comprising:
making a site of the PSEN1 gene deficient by a genome-editing technology, the site including at least a 5′- or 3′-splice site of exon 9 in the PSEN1 gene and being related to splicing of exon 9.

4. The method as set forth in claim 3, wherein
the site including at least the 5′- or 3′-splice site of exon 9 in the PSEN1 gene and being related to splicing of exon 9 is made deficient in an ovum.

5. The method as set forth in claim 3, wherein
the non-human primate is a marmoset.

6. A brain of the non-human primate model animal of Alzheimer's disease recited in claim 1.

7. A somatic cell of the non-human primate model animal of Alzheimer's disease recited in claim 1.


SEQUENCE LISTING (21 sequences)

SEQ ID NO: 1 - DNA, 8174 bp, artificial sequence
  vector sequence for Left TALEN
agaaggggctcccgcacgcgcctgcattgattaagcggaccaacagaaggatccccgagaggacatcacatcgagtggcaggttcccaactcgtgaagagtgaacttgaggagaaaaagtcggagctgcggcacaaattgaaatacgtaccgcatgaatacatcgaacttatcgaaattgctaggaactcgactcaagacagaatccttgagatgaaggtaatggagttctttatgaaggtttatggataccgagggaagcatctcggtggatcacgaaaacccgacggagcaatctatacggtggggagcccgattgattacggagtgatcgtcgacacgaaagcctacagcggtgggtacaatcttcccatcgggcaggcagatgagatgcaacgttatgtcgaagaaaatcagaccaggaacaaacacatcaatccaaatgagtggtggaaagtgtatccttcatcagtgaccgagtttaagtttttgtttgtctctgggcatttcaaaggcaactataaggcccagctcacacggttgaatcacattacgaactgcaatggtgcggttttgtccgtagaggaactgctcattggtggagaaatgatcaaagcgggaactctgacactggaagaagtcagacgcaagtttaacaatggcgagatcaatttccgctcataaaaaatcagcctcgactgtgccttctagttgccagccatctgttgtttgcccctcccccgtgccttccttgaccctggaaggtgccactcccactgtcctttcctaataaaatgaggaaattgcatcacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtttccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcattaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatcatttagaataaattttgtgtcgcccttaattgtgagcggataacaattacgagcttcatgcacagtggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggaagcttcttgttctttttgcagaagctcagaataaacgctcaactttggcctcgaggccaccatggactataaggaccacgacggagactacaaggatcatgatattgattacaaagacgatgacgataagatggccccaaagaagaagcggaaggtcggtatccacggagtcccagcagccgtagatttgagaactttgggatattcacagcagcagcaggaaaagatcaagcccaaagtgaggtcgacagtcgcgcagcatcacgaagcgctggtgggtcatgggtttacacatgcccacatcgtagccttgtcgcagcaccctgcagcccttggcacggtcgccgtcaagtaccaggacatgattgcggcgttgccggaagccacacatgaggcgatcgtcggtgtggggaaacagtggagcggagcccgagcgcttgaggccctgttgacggtcgcgggagagctgagagggcctccccttcagctggacacgggccagttgctgaagatcgcgaagcggggaggagtcacggcggtcgaggcggtgcacgcgtggcgcaatgcgctcacgggagcacccctcaacctgaccccagaccaggttgtggccatcgccagcaacataggtggcaagcaggccctcgaaaccgtccagagactgttaccggttctctgccaggaccacggcctgaccccggaacaggtggttgcaatcgcgtcacacgatgggggaaagcaggccctagaaaccgttcagcgactcctgcccgtcctgtgccaggcccacggcctgaccccggaccaggtggttgcaatcgcgtcacacgatgggggaaagcaggccctagaaaccgttcagcgactcctgcccgtcctgtgccaggcccacggcctgacccccgcccaggttgtcgctattgctagtaacggcggaggcaaacaggcgctggaaacagttcagcgcctcttgccggtcttgtgtcaggaccacggcctgaccccagaccaagttgtcgcgattgcaagcaacaacggaggcaaacaagccttagaaacagtccagagattgttgccggtgctgtgccaagaccacggcctgaccccagaacaagttgtcgcgattgcaagcaacaacggaggcaaacaagccttagaaacagtccagagattgttgccggtgctgtgccaagcccacggcctgaccccagaccaggttgtggccatcgccagcaacataggtggcaagcaggccctcgaaaccgtccagagactgttaccggttctctgccaggcccacggcctgaccccagcccaggttgtggccatcgccagcaacataggtggcaagcaggccctcgaaaccgtccagagactgttaccggttctctgccaggaccacggcctgacccccgaccaggttgtcgctattgctagtaacggcggaggcaaacaggcgctggaaacagttcagcgcctcttgccggtcttgtgtcaggaccacggcctgacccccgaacaggttgtcgctattgctagtaacggcggaggcaaacaggcgctggaaacagttcagcgcctcttgccggtcttgtgtcaggcccacggcctgacccccgaccaggttgtcgctattgctagtaacggcggaggcaaacaggcgctggaaacagttcagcgcctcttgccggtcttgtgtcaggcccacggcctgacccccgcccaggttgtcgctattgctagtaacggcggaggcaaacaggcgctggaaacagttcagcgcctcttgccggtcttgtgtcaggaccacggcctgaccccagaccaagttgtcgcgattgcaagcaacaacggaggcaaacaagccttagaaacagtccagagattgttgccggtgctgtgccaagaccacggcctgacccccgaacaggttgtcgctattgctagtaacggcggaggcaaacaggcgctggaaacagttcagcgcctcttgccggtcttgtgtcaggcccacggcctgaccccggaccaggtggttgcaatcgcgtcacacgatgggggaaagcaggccctagaaaccgttcagcgactcctgcccgtcctgtgccaggcccacggcctgacccccgcccaggttgtcgctattgctagtaacggcggaggcaaacaggcgctggaaacagttcagcgcctcttgccggtcttgtgtcaggaccacggcctgacgcctgagcaggtagtggctattgcatccaacggagggggcagacccgcactggagtcaatcgtggcccagctttcgaggccggaccccgcgctggccgcactcactaatgatcatcttgtagcgctggcctgcctcggcggacgacccgccttggatgcggtga

SEQ ID NO: 2 - DNA, 8174 bp, artificial sequence
  vector sequence for Right TALEN
agaaggggctcccgcacgcgcctgcattgattaagcggaccaacagaaggatccccgagaggacatcacatcgagtggcaggttcccaactcgtgaagagtgaacttgaggagaaaaagtcggagctgcggcacaaattgaaatacgtaccgcatgaatacatcgaacttatcgaaattgctaggaactcgactcaagacagaatccttgagatgaaggtaatggagttctttatgaaggtttatggataccgagggaagcatctcggtggatcacgaaaacccgacggagcaatctatacggtggggagcccgattgattacggagtgatcgtcgacacgaaagcctacagcggtgggtacaatcttcccatcgggcaggcagatgagatgcaacgttatgtcgaagaaaatcagaccaggaacaaacacatcaatccaaatgagtggtggaaagtgtatccttcatcagtgaccgagtttaagtttttgtttgtctctgggcatttcaaaggcaactataaggcccagctcacacggttgaatcacattacgaactgcaatggtgcggttttgtccgtagaggaactgctcattggtggagaaatgatcaaagcgggaactctgacactggaagaagtcagacgcaagtttaacaatggcgagatcaatttccgctcataaaaaatcagcctcgactgtgccttctagttgccagccatctgttgtttgcccctcccccgtgccttccttgaccctggaaggtgccactcccactgtcctttcctaataaaatgaggaaattgcatcacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtttccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcattaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatcatttagaataaattttgtgtcgcccttaattgtgagcggataacaattacgagcttcatgcacagtggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggaagcttcttgttctttttgcagaagctcagaataaacgctcaactttggcctcgaggccaccatggactataaggaccacgacggagactacaaggatcatgatattgattacaaagacgatgacgataagatggccccaaagaagaagcggaaggtcggtatccacggagtcccagcagccgtagatttgagaactttgggatattcacagcagcagcaggaaaagatcaagcccaaagtgaggtcgacagtcgcgcagcatcacgaagcgctggtgggtcatgggtttacacatgcccacatcgtagccttgtcgcagcaccctgcagcccttggcacggtcgccgtcaagtaccaggacatgattgcggcgttgccggaagccacacatgaggcgatcgtcggtgtggggaaacagtggagcggagcccgagcgcttgaggccctgttgacggtcgcgggagagctgagagggcctccccttcagctggacacgggccagttgctgaagatcgcgaagcggggaggagtcacggcggtcgaggcggtgcacgcgtggcacaatgcgctcacgggagcacccctcaacctgaccccagaccaggttgtggccatcgccagcaacataggtggcaagcaggccctcgaaaccgtccagagactgttaccggttctctgccaggaccacggcctgacccccgaacaggttgtcgctattgctagtaacggcggaggcaaacaggcgctggaaacagttcagcgcctcttgccggtcttgtgtcaggcccacggcctgacccccgaccaggttgtcgctattgctagtaacggcggaggcaaacaggcgctggaaacagttcagcgcctcttgccggtcttgtgtcaggcccacggcctgaccccggcccaggtggttgcaatcgcgtcacacgatgggggaaagcaggccctagaaaccgttcagcgactcctgcccgtcctgtgccaggaccacggcctgaccccagaccaggttgtggccatcgccagcaacataggtggcaagcaggccctcgaaaccgtccagagactgttaccggttctctgccaggaccacggcctgaccccggaacaggtggttgcaatcgcgtcacacgatgggggaaagcaggccctagaaaccgttcagcgactcctgcccgtcctgtgccaggcccacggcctgaccccggaccaggtggttgcaatcgcgtcacacgatgggggaaagcaggccctagaaaccgttcagcgactcctgcccgtcctgtgccaggcccacggcctgaccccagcccaggttgtggccatcgccagcaacataggtggcaagcaggccctcgaaaccgtccagagactgttaccggttctctgccaggaccacggcctgaccccagaccaggttgtggccatcgccagcaacataggtggcaagcaggccctcgaaaccgtccagagactgttaccggttctctgccaggaccacggcctgaccccggaacaggtggttgcaatcgcgtcacacgatgggggaaagcaggccctagaaaccgttcagcgactcctgcccgtcctgtgccaggcccacggcctgaccccggaccaggtggttgcaatcgcgtcacacgatgggggaaagcaggccctagaaaccgttcagcgactcctgcccgtcctgtgccaggcccacggcctgaccccagcccaggttgtggccatcgccagcaacataggtggcaagcaggccctcgaaaccgtccagagactgttaccggttctctgccaggaccacggcctgaccccggaccaggtggttgcaatcgcgtcacacgatgggggaaagcaggccctagaaaccgttcagcgactcctgcccgtcctgtgccaggaccacggcctgaccccagaacaggttgtggccatcgccagcaacataggtggcaagcaggccctcgaaaccgtccagagactgttaccggttctctgccaggcccacggcctgaccccggaccaggtggttgcaatcgcgtcacacgatgggggaaagcaggccctagaaaccgttcagcgactcctgcccgtcctgtgccaggcccacggcctgaccccggcccaggtggttgcaatcgcgtcacacgatgggggaaagcaggccctagaaaccgttcagcgactcctgcccgtcctgtgccaggaccacggcctgacgcctgagcaggtagtggctattgcatccaacatagggggcagacccgcactggagtcaatcgtggcccagctttcgaggccggaccccgcgctggccgcactcactaatgatcatcttgtagcgctggcctgcctcggcggacgacccgccttggatgcggtga

SEQ ID NO: 3 - DNA, 20 bp, artificial sequence
  primer sequence
acccgcgactccctattatt

SEQ ID NO: 4 - DNA, 20 bp, artificial sequence
  primer sequence
tgccttgactgtattgttgg

SEQ ID NO: 5 - DNA, 20 bp, artificial sequence
  primer sequence
tacctccctgaatggactgc

SEQ ID NO: 6 - DNA, 20 bp, artificial sequence
  primer sequence
tggttgtgttccagtctcca

SEQ ID NO: 7 - DNA, 20 bp, artificial sequence
  primer sequence
ggtccacttcgtatgctggt

SEQ ID NO: 8 - DNA, 20 bp, artificial sequence
  primer sequence
ggctgttgctgaggctttac

SEQ ID NO: 9 - DNA, 51 bp, Callithrix jacchus
tacctggaattttgtctttctcaacagcaacaatggtgtggttggtgaata

SEQ ID NO: 10 - DNA, 34 bp, Callithrix jacchus
tttgtctttctcaacagcaacaatggtgtggttg

SEQ ID NO: 11 - DNA, 31 bp, artificial sequence
  3′-splice site of exon 9 (-3bp) (5 cell)
tttgtctttctcaccaacaatggtgtggttg

SEQ ID NO: 12 - DNA, 31 bp, artificial sequence
  3′-splice site of exon 9 (-3bp) (Morula-1)
tttgtctttctcaacaacaatggtgtggttg

SEQ ID NO: 13 - DNA, 29 bp, artificial sequence
  3′-splice site of exon 9 (-6bp) (PSEN1-delta E9)
tttgtctttctcaacaaatggtgtggttg

SEQ ID NO: 14 - DNA, 34 bp, artificial sequence; identical to SEQ ID NO: 10
  3′-splice site of exon 9 (intact) (PSEN1-delta E9)

SEQ ID NO: 15 - DNA, 59 bp, Callithrix jacchus
tgcttacctggaattttgtctttctcaacagcaacaatggtgtggttggtgaatatggc

SEQ ID NO: 16 - DNA, 53 bp, artificial sequence
  3′-splice site of exon 9 (-6bp) (I774gmF)
tgcttacctggaattttgtctttctcaacaatggtatggttggtgaatatggc

SEQ ID NO: 17 - DNA, 59 bp, artificial sequence; identical to SEQ ID NO: 15
  3′-splice site of exon 9 (intact) (I774gmF)

SEQ ID NO: 18 - DNA, 53 bp, artificial sequence; identical to SEQ ID NO: 16
  3′-splice site of exon 9 (-6bp) (I943gmM)

SEQ ID NO: 19 - DNA, 59 bp, artificial sequence; identical to SEQ ID NO: 15
  3′-splice site of exon 9 (intact) (I943gmM)

SEQ ID NO: 20 - DNA, 50 bp, artificial sequence
  3′-splice site of exon 9 (-9bp) (I949gmM)
tgcttacctggaattttgtctttctcaatggtatggttggtgaatatggc

SEQ ID NO: 21 - DNA, 59 bp, artificial sequence; identical to SEQ ID NO: 15
  3′-splice site of exon 9 (intact) (I949gmM)